(19) United States
(12) Patent Application Publication
(10) Pub. No.: US 2023/0204584 A1
(43) Pub. Date: Jun. 29, 2023

(54) PROTEIN SIGNATURE FOR SCREENING GENERAL POPULATION FOR COLORECTAL CANCER AND/OR PRE-CANCEROUS STAGE THEREOF

(71) Applicant: Advanced Marker Discovery S.L., Valladolid (ES)

(72) Inventors: Ana Carmen Martín Rodríguez, Valladolid (ES); Lourdes Castillo García, Valladolid (ES); Carmen Monsalve Hernando, Valladolid (ES); Rosa Pérez Palacios, Valladolid (ES); Rocío Arroyo Arranz, Valladolid (ES)

(21) Appl. No.: 17/906,597

(22) PCT Filed: Mar. 18, 2021
(86) PCT No.: PCT/EP2021/056984

§ 371 Date: Sep. 16, 2022

(30) Foreign Application Priority Data
Mar. 19, 2020 (EP) 20382204.4
Mar. 19, 2020 (EP) 20382205.1

(51) Int. Cl. G01N 33/574

(52) U.S. Cl. CPC G01N 33/57419; G01N 33/57484

(57) ABSTRACT

The present invention refers to an in vitro method for the diagnosis of colorectal cancer and/or pre-cancerous stage thereof.

### FIELD OF THE INVENTION

The present invention can be included in the medical field. Particularly, the present invention refers to an in vitro method for screening general population for colorectal cancer and/or pre-cancerous stage thereof.

### STATE OF THE ART

Colorectal cancer (CRC) (also known as colon cancer, rectal cancer, or bowel cancer) is the development of cancer in the colon or rectum (parts of the large intestine). The vast majority of colorectal cancers is adenocarcinomas. This is because the colon has numerous glands within the tissue. When these glands undergo several changes at the genetic level, they proceed in a predictable manner as they move from benign to an invasive, malignant colon cancer. The adenomas of the colon, particularly advanced colorectal adenoma (AA), are a benign version of the malignant adenocarcinomas but still with malignant potential if not removed (they are usually removed because of their tendency to become malignant and to lead to colon cancer).

Screening is an effective way for preventing and decreasing deaths from colorectal cancer. The best known and most frequently used screening test for colorectal cancer is called Fecal Immunochemical Test (FIT). FIT detects blood in the stool samples which can be a sign of pre-cancer or cancer. If abnormal results are obtained, usually a colonoscopy is recommended which allows the physician to look at the inside of the colon and rectum to make a diagnosis. During colonoscopy, small polyps may be removed if found. If a large polyp or tumor is found, a biopsy may be performed to check if it is cancerous. The gastroenterologist uses a colonoscopy to find and remove these adenomas and polyps to prevent them from continuing to acquire genetic changes that will lead to an invasive adenocarcinoma.

Although, as explained above, FIT is nowadays used for screening colorectal cancer, it is important to note that FIT offers a low sensitivity for AA (around 20-30% depending on literature) which means that most of said kind of patients can be wrongly classified as not having the disease. Consequently, FIT is not able to identify adenomas due to its low sensitivity. Moreover, since FIT uses stool samples, it offers a low compliance. On the other hand, the colonoscopy is an invasive technique wherein the most severe complication generally is the gastrointestinal perforation. Moreover, colonoscopy is nowadays a procedure involving anesthesia, and the laxatives which are usually administered during the bowel preparation for colonoscopy are associated with several digestive problems.

It is important to note that the methods used today for screening general population at risk of suffering for CRC or AA are associated with a high rate of false positives. Consequently, a high amount of unnecessary follow-up colonoscopies is nowadays performed.

The present invention offers a clear solution to the problems cited above because it is focused on an in vitro method for identifying or screening human subjects at risk of suffering from colorectal cancer or colorectal adenomas (particularly advanced colorectal adenomas), departing from the concentration level of protein biomarkers isolated from minimally-invasive samples such as blood, serum or plasma. Since the method of the invention is based on blood, serum or plasma samples, it is expected to improve compliance to colorectal cancer screening. Moreover, the method of the invention offers high sensitivity and specificity, which means that it is a strong and cost-effective method for the detection of both colorectal cancer and colorectal adenomas.

### DESCRIPTION OF THE INVENTION

### Brief Description of the Invention

The present invention refers to an in vitro method for screening general population for colorectal cancer and/or advanced colorectal adenomas, departing from the concentration level of protein biomarkers isolated from minimally-invasive samples such as blood, serum or plasma. The method of the invention offers high sensitivity and specificity, which means that it is a strong and cost-effective method for the detection of both colorectal cancer and colorectal adenomas.

Since the method of the invention has higher sensitivity and specificity as compared to the method used today (FIT) for screening general population at risk of suffering from CRC or AA, it is associated with a lower percentage of false positives. Consequently, the method described in the present invention clearly helps in reducing the number of follow-up colonoscopies, thus improving the way that the patients are nowadays screened. Once the method of the invention is performed, if it is determined that the patients might be suffering from colorectal cancer and/or precancerous stage, the result is confirmed by colonoscopy. However, if it is not determined that the patient might be suffering from colorectal cancer and/or precancerous stage, there is no need to perform a colonoscopy and routine testing with the method of the invention defined below is recommended.

Particularly, the first embodiment of the present invention refers to an in vitro method (hereinafter “method of the invention”) for screening general population for colorectal cancer and/or a pre-cancerous stage thereof which comprises: a) Measuring the concentration level of at least TFF3, in a minimally invasive biological sample obtained from the subject, and b) wherein if a deviation or variation of the concentration level is identified, as compared with the reference concentration level measured in healthy control subjects, this is indicative that the subject is suffering from colorectal cancer or a precancerous stage thereof.

Alternatively, the first embodiment of the present invention refers to an in vitro method for detecting at least TFF3 in a test sample from a asymptomatic human subject at risk of developing colorectal cancer and/or a pre-cancerous stage thereof, comprising: detecting whether the protein biomarker is present in a minimally invasive sample obtained from the subject by contacting the plasma sample with an antibody directed against said protein biomarker and detecting binding between the protein and the antibody.

In a preferred embodiment, the present invention refers to an in vitro method for screening general population for colorectal cancer and/or a pre-cancerous stage thereof which comprises: a) Measuring the concentration level of at least the combination [TFF3 and Flt3L], in a minimally invasive biological sample obtained from the subject, and b) wherein if a deviation or variation of the concentration level of at least the combination [TFF3 and Flt3L] is identified, as compared with the reference concentration level measured in healthy control subjects, this is indicative that the subject is suffering from colorectal cancer or a precancerous stage thereof.

In this regard, it is important to consider that the most reliable biomarker identified in the present invention for screening general population for colorectal cancer or a precancerous stage thereof is TFF3 (see Table 3). Moreover, the present invention also offers signatures for CRC screening always comprising TFF3 or [TFF3 and Flt3L] which show an AUC>0.8), for example: [TFF3 and Flt3L and HGFR], [TFF3 and Flt3L and IGFBP2], [TFF3 and Flt3L and CD147], [TFF3 and Flt3L and CD163], [TFF3 and Flt3L and CYFRA21-1], [TFF3 and Flt3L and HGFR and IGFBP2], [TFF3 and Flt3L and HGFR and CD147], [TFF3 and Flt3L and IGFBP2 and CD147], [TFF3 and Flt3L and CD163 and IGFBP2], [TFF3 and Flt3L and CD163 and HGFR], [TFF3 and Flt3L and CD163 and CD147], [TFF3 and Flt3L and CYFRA21-1 and CD147], [TFF3 and Flt3L and CYFRA21-1 and IGFBP2], [TFF3 and Flt3L and CD163 and CYFRA21-1], [TFF3 and Flt3L and HGFR and CYFRA21-1]. Consequently, any of the signatures comprising TFF3 or [TFF3 and Flt3L] could be effectively used according to the present invention, since they offer an AUC value above 0.8 for CRC screening and a good performance for of AA screening.

In a particularly preferred embodiment, the method of the invention comprises a) measuring the concentration level of at least the combination [TFF3 and Flt3L and HGFR], [TFF3 and Flt3L and IGFBP2], [TFF3 and Flt3L and CD147], [TFF3 and Flt3L and CD163], [TFF3 and Flt3L and CYFRA21-1], [TFF3 and Flt3L and HGFR and IGFBP2], [TFF3 and Flt3L and HGFR and CD147], [TFF3 and Flt3L and IGFBP2 and CD147], [TFF3 and Flt3L and CD163 and IGFBP2], [TFF3 and Flt3L and CD163 and HGFR], [TFF3 and Flt3L and CD163 and CD147], [TFF3 and Flt3L and CYFRA21-1 and CD147], [TFF3 and Flt3L and CYFRA21-1 and IGFBP2], [TFF3 and Flt3L and CD163 and CYFRA21-1], [TFF3 and Flt3L and HGFR and CYFRA21-1], and b) wherein if a deviation or variation of the concentration level of at least one of the above cited combination of biomarkers is identified, as compared with the reference concentration level measured in healthy control subjects, this is indicative that the subject is suffering from colorectal cancer or a precancerous stage thereof. The second embodiment of the present invention refers to the in vitro use of any of the above cited biomarkers or signatures for screening general population for colorectal cancer or a precancerous stage thereof.

According to the method of the invention, after measuring the concentration level of any of the above cited combinations of biomarkers, a score value is obtained for the signature and this score value is compared with a threshold value which defines the diagnostic rule. If this score value is higher than the threshold, then the corresponding sample is classified as a positive sample, which is an indication that the patient might be suffering from colorectal cancer and/or pre-cancerous stage thereof. The threshold value has been defined in order to optimize sensitivity and specificity values. Consequently, in a preferred embodiment, the method of the invention comprises: a) Measuring the concentration level of any of the above cited combinations of biomarkers, in a biological sample obtained from the subject, b) processing the concentration values in order to obtain a risk score and c) wherein if a deviation or variation of the risk score value obtained for any of the above cited combinations of biomarkers is identified, as compared with a reference value, this is indicative that the subject is suffering from colorectal cancer and/or a pre-cancerous stage.

The third embodiment of the present invention refers to a kit of parts comprising reagents for determining the concentration level of any of the above cited biomarkers or signatures. In a preferred embodiment, the present invention refers to the in vitro use of a kit comprising reagents for the determination of the concentration level of any of the above cited biomarkers or signatures for screening general population for colorectal cancer or a precancerous stage thereof.

In a preferred embodiment, the pre-cancerous stage of colorectal cancer is advanced colorectal adenoma.

In a preferred embodiment, the method of the invention is confirmed by an image technique, preferably colonoscopy.

In a preferred embodiment the present invention refers to an in vitro method for screening general population for colorectal cancer or a precancerous stage thereof, said method comprising: a) obtaining a minimally invasive sample from a human patient; and b) detecting whether any of the above cited protein biomarkers or signatures are present in the sample by contacting the plasma sample with an antibody directed against said protein biomarkers or signatures and detecting binding between the proteins and the antibody.

The last embodiment of the present invention refers to a method for screening general population for colorectal cancer or a precancerous stage thereof and treating those subjects identified as suffering from colorectal cancer or a pre-cancerous stage thereof, which comprises: a) obtaining a minimally invasive sample from a human patient; b) detecting whether any of the above cited protein biomarkers or signatures are present in the sample; c) diagnosing the patient with colorectal cancer or a pre-cancerous stage thereof when the presence of said protein biomarkers or signatures in the sample is detected; and d) performing a colonoscopy to the patient and removing the colorectal cancer or polyps afterwards.

In a preferred embodiment, the present invention is a computer-implemented invention, wherein a processing unit (hardware) and a software are configured to:
Receive the concentration level values of any of the above cited biomarkers or signatures,
Process the concentration level values received for finding substantial variations or deviations, and
Provide an output through a terminal display of the variation or deviation of the concentration level, wherein the variation or deviation of the concentration level indicates that the subject may be suffering from colorectal cancer and, optionally, this result is confirmed by colonoscopy.

For the purpose of the present invention the following terms are defined:
The term “screening” (sometimes termed medical surveillance) refers to a medical procedure performed on members (subjects) of a defined asymptomatic population or population subgroup to assess the likelihood of their members having a particular disease. It is important to note that, according to the World Health Organization, although both early diagnosis and screening are two components of the concept “early detection”, there are important differences between them. While early diagnosis focuses on detecting symptomatic patients as early as possible (diagnostic tests are used to find out the cause of the symptoms), screening consists of testing healthy individuals (who don't have symptoms) to identify those having cancer before any symptoms appear: https://www.euro.who.int/en/health-topics/noncommunicable-diseases/cancer/policy/screening-and-early-detection
The expression “general population” refers to asymptomatic population.
The term “colorectal cancer” is a medical condition characterized by cancer of cells of the intestinal tract below the small intestine (i.e., the large intestine (colon), including the cecum, ascending colon, transverse colon, descending colon, sigmoid colon, and rectum).
The expression “colorectal adenoma” refers to adenomas of the colon, also called adenomatous polyps, which is a benign and pre-cancerous stage of the colorectal cancer but still with high risk of progression to colorectal cancer.
The expression “advanced colorectal adenoma” refers to adenomas having a size of at least 10 mm or histologically having high grade dysplasia or a villous component higher than 20%.
The expression “minimally-invasive biological sample” refers to any sample which is taken from the body of the patient without the need of using harmful instruments, other than fine needles used for taking the blood from the patient, and consequently without being harmfully for the patient. Specifically, minimally-invasive biological sample refers in the present invention to: blood, serum, or plasma samples.
The expression “reference concentration level measured in healthy control subjects”, refer to a “reference value” of the concentration level of the proteins. If a deviation of the concentration level of the proteins is determined with respect to said “reference concentration level measured in healthy control subjects”, this is an indication of colorectal cancer or pre-cancerous stage thereof. Particularly, if the concentration level of the biomarkers or signatures of the present invention are significantly higher or lower with respect to said “reference value” this is an indication of colorectal cancer or pre-cancerous stage thereof.
The expression “risk score” refers to a risk value obtained after processing one or more concentration values into a single value (or risk value), which represents the probability of disease for the individual. This risk value will be compared with a reference value to evaluate if the patient might be suffering from colorectal cancer and/or pre-cancerous stage thereof.
A “reference value” can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the levels of the biomarkers in a group of reference, one can use algorithmic analysis for the statistic treatment of the measured concentrations of biomarkers in biological samples to be tested, and thus obtain a classification standard having significance for sample classification. The ROC curve is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis/prognosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is good. When AUC is higher than 0.9, the accuracy is quite high. This algorithmic method is preferably done with a computer. Existing software or systems in the art may be used for the drawing of the ROC curve, such as: MedCalc medical statistical software, SPSS.
By “comprising” it is meant including, but not limited to, whatever follows the word “comprising”. Thus, use of the term “comprising” indicates that the listed elements are required or mandatory, but that other elements are optional and may or may not be present.
By “consisting of” it is meant “including, and limited to”, whatever follows the phrase “consisting of”. Thus, the phrase “consisting of” indicates that the listed elements are required or mandatory, and that no other elements may be present.

For the purpose of the present invention the following proteins are identified according to Uniprot data base:

| | | Uniprot |
|---|---|---|
| Abbreviation | Protein Name | ID. |
| TFF3 | Trefoil factor 3 | Q07654 |
| CYFRA21-1 | Cytokeratin fragment antigen 21-1 | N/A |
| Flt3L | Fms-related tyrosine kinase 3 ligand | P49771 |
| AREG | Amphiregulin | P15514 |
| CASP4 | Caspase-4 | P49662 |
| IGFBP2 | Insulin-like growth factor-binding protein 2 | P18065 |
| ADAMDEC1 | ADAM DEC1 | O15204 |
| DKK3 | Dickkopf-related protein 3 | Q9UBP4 |
| FASL | Tumor necrosis factor ligand superfamily | P48023 |
| | member 6 | |
| DCSIGNR | C-type lectin domain family 4 member M | Q9H2X3 |
| ErbB4 | Receptor tyrosine-protein kinase erbB-4 | Q15303 |
| CD163 | Scavenger receptor cysteine-rich type 1 | Q86VB7 |
| | protein M130 | |
| HGFR | Hepatocyte growth factor receptor | P08581 |
| CEA | Carcinoembryonic antigen | P06731 |
| CD147 | Basigin | P35613 |
| PKM2 | Pyruvate kinase PKM | P14618 |

### BRIEF DESCRIPTION OF THE FIGURES

FIG. 1. Receiver-operating-characteristic (ROC) curve in colorectal cancer for: A) [TFF3 and Flt3L]. Area Under Curve (AUC)=0.8672. B) [TFF3 and Flt3L and HGFR]. Area Under Curve (AUC)=0.8672. X axis represents Specificity. Y axis represents Sensitivity.

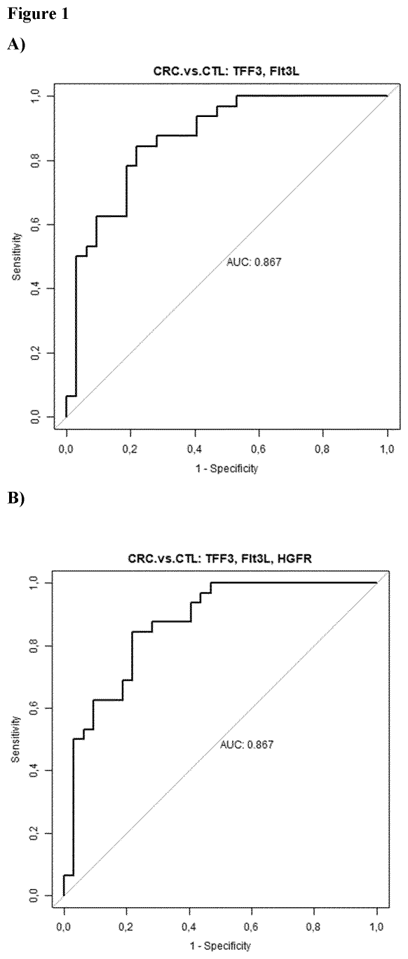

FIG. 2. Receiver-operating-characteristic (ROC) curve in advanced colorectal adenoma for [TFF3/Flt3L/CD163/ADAMDEC1]. Area Under Curve (AUC)=0.6815.

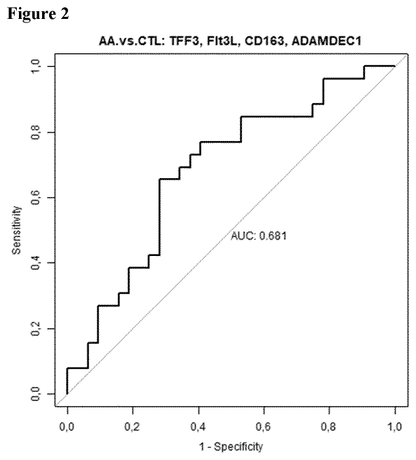

X axis represents Specificity. Y axis represents Sensitivity.

### DETAILED DESCRIPTION OF THE INVENTION

### Example 1. Material and Methods

### Example 1.1. Population of Study

The population of study was Spanish screening population. This means asymptomatic average risk subjects between age 50-75 referred to colonoscopy by a population screening program. Due to the low incidence of CRC in screening population, some CRC cases are patients already diagnosed with CRC that were scheduled for surgery (blood samples were obtained before colonoscopy or surgery resection). Subjects who have developed another type of cancer in the 5 years prior to their participation in the study or patients who have previously received chemotherapy or radiotherapy, or patients diagnosed with Non-Advanced Adenomas, Familiar Adenomatous Polyposis or Lynch Syndrome, Inflammatory Bowel Disease, or inadequate intestinal preparation for colonoscopy or subjects who have undergone colonoscopy/polypectomy in the previous 5 years were excluded from the study.

At the end, a total of 180 subjects from six Spanish hospitals (Hospital de Burgos, Hospital de Vigo, Hospital de Alicante, Hospital de Ourense, Instituto Valenciano de Oncologia, and Hospital de Bellvitge) were prospectively included in this study: 120 patients newly diagnosed with sporadic colorectal neoplasia (60 with CRC and 60 with AA) and 60 healthy individuals.

The results were validated in a cohort of 92 subjects from four Spanish hospitals (Hospital de Burgos, Hospital de Vigo, Hospital de Ourense, Instituto Valenciano de Oncologia): 59 patients diagnosed with sporadic colorectal neoplasia (32 with CRC and 27 with AA) and 33 healthy individuals.

All subjects without personal history of any cancer and with a recent colonoscopy confirming the lack of colorectal neoplastic lesions. Patients with AA were those with adenomas having a size of at least 10 mm or histologically having high grade dysplasia or >20% villous component. The characteristics of participants are shown in Table 1 and Table 2. Blood samples were collected prior to endoscopy or surgery in all individuals.

TABLE 1
Clinical-pathological characteristics of the study cohort 180

| Cases | Control (CTL) | AA | CRC | | TOTAL | |
|---|---|---|---|---|---|---|
| Mean age (SD) | 58 (34-74) | 62 (36-81) | 64.7 | (38-86) | 61.6 | (34-86) |

GENDER

| | | | | | | |
|---|---|---|---|---|---|---|
| Male | 30 (50%) | 36 (60%) | 31 | (51.6%) | 97 | (53.8%) |
| Female | 30 (50%) | 24 (40%) | 29 | (48.4%) | 83 | (46.2%) |

COLORECTAL FEATURES
TNM stage

| | |
|---|---|
| I | 13 |
| II | 13 |
| III | 18 |
| IV | 12 |
| Unknown | 4 |

Location

| | |
|---|---|
| Ascending colon and cecum | 17 |
| Descending colon and sigma | 16 |
| Transverse | 1 |
| Rectum | 21 |
| Hepatic flexure | 5 |

ADVANCED COLORECTAL ADENOMA
FEATURES

| | |
|---|---|
| Size => 10 mm | 49 |
| Small AA (<=15 mm) | 29 |
| Big AA (>15 mm) | 23 |
| Unknown | 8 |
| Mean size (mm) (SD) | 17 |
| No. AAs Mean (SD) | 1.57 |

High-grade dysplasia

| | |
|---|---|
| Yes | 21 |
| No | 30 |
| Unknown | 9 |

Villous component

| | |
|---|---|
| Yes | 17 |
| No | 33 |
| Unknown | 10 |

TABLE 2
Clinical-pathological characteristics of the study cohort 92

| Cases | Control (CTL) | AA | CRC | TOTAL |
|---|---|---|---|---|
| Mean age (SD) | 64 (44-36) | 65.3 (39-88) | 72 (54-85) | 67 (39-88) |

GENDER

| | | | | |
|---|---|---|---|---|
| Male | 16 (48.5%) | 16 (59.3%) | 17 (53%) | 49 (53.2%) |
| Female | 17 (51.5%) | 11 (40.7%) | 15 (47%) | 43 (46.8%) |

COLORECTAL FEATURES
TNM stage

| | |
|---|---|
| I | 3 |
| II | 9 |
| III | 9 |
| IV | 7 |
| Unknown | 4 |

Location

| | |
|---|---|
| Ascending colon and cecum | 11 |
| Descending colon and sigma | 11 |
| Transverse | 3 |
| Rectum | 6 |
| Unknown | 1 |

ADVANCED COLORECTAL ADENOMA
FEATURES

| | |
|---|---|
| Size => 10 mm | 22 |
| Small AA (<=15 mm) | 15 |
| Big AA (>15 mm) | 11 |
| Mean size (mm) (SD) | 18.5 |
| No. AAs Mean (SD) | 2.2 |
| Unknown | 1 |

High-grade dysplasia

| | |
|---|---|
| Yes | 9 |
| No | 16 |
| Unknown | 2 |

Villous component

| | |
|---|---|
| Yes | 13 |
| No | 12 |
| Unknown | 2 |

The study was approved by the Institutional Ethics Committee of each Hospital, and written informed consent was obtained from all participants in accordance with the Declaration of Helsinki.

### Example 1.2. Sample Preparation

Ten mL of whole blood from each participant were collected in EDTA K2 containing tubes. Blood samples were placed at 4° C. until plasma separation. Samples were centrifuged at 1,600×g for 10 min at 4° C. to spin down blood cells, and plasma was transferred into new tubes, followed by further centrifugation at 16,000×g for 10 minutes at 4° C. to completely remove cellular components.

Blood samples were obtained before colonoscopy or surgery resection.

### Example 1.3. Molecular Analysis

The concentration of the biomarkers in plasma samples was established using commercial ELISA (Enzyme-linked immunosorbent assay) and CLIA (Chemiluminescence immunoassay) test and following their corresponding instruction manual. HGFR, ErbB4, CEA, CD163, DKK3, IGFBP2, and TFF3 was analyzed with ELISA kit from Cloud clone Corp. Level of CD147, Flt3L, FasL and Casp4, was measured using ELISA Kit form Elabscience. In the case of the DCSINGR, ELISA kit from RayBio was used. PKM2 were analyzed with ELISA kit from Aviva and ADAMDEC1 with ELISA kit from Cusabio. Related to CLIA test, CYFRA21-1 y AREG was analyzed with CLIA test from Cloud Clone Corp.

### Example 1.4. Data Quantification

For the protein quantification step the samples were processed with the corresponding kit (ELISA/CLIA) and distributed in experimental plates. Each plate contained also control data used to construct a standard curve. Fluorescence data obtained from each run (expressed as integer numbers) have been background corrected for each sample and quantified using a standard curve generated using a 2-degree polynomial regression model.

### Example 1.5. Statistical Analysis

Three groups of individuals were considered in the analysis. CRC (Individuals diagnosed with colorectal cancer), AA (Individuals diagnosed with advanced adenoma) and CTL (Individuals with no disease).

Raw quantification data have been transformed by applying square root function, and then centering and scaling so that, after the transformation, each protein measure has mean 0 and standard deviation 1.

To deal with non-normality issues, Wilcoxon rank-sum test was used to compare either CRC cases or AA cases against CTL individuals.

Diagnostic performance for the individual proteins and some of their combinations has been assessed by their receiver operating characteristic (ROC) curves, and the area under the ROC curves (AUC). Moreover, sensitivities, specificities, positive predictive and negative predictive values (PPV and NPV) for the different tests were calculated at the optimal cutoff point defined by the best Youden's Index (or equivalently, the point of the ROC that maximizes the sum of sensitivity and specificity).

Scores used for deriving the ROC-AUCs and the rest of performance values were obtained using univariate logistic regression model for the individual proteins and multivariate logistic regression models for the different combination of proteins considered. 95% CI for the AUCs was obtained with the DeLong methodology both in individual markers and combination of them.

All the individual performance metrics obtained from the previous analysis are summarized in Table 3 and Table 4.

Moreover, multivariate analysis has been carried out to explore if combinations of proteins improve the performance of individual markers. We have used multivariate logistic regression to fit models with all possible combinations of two, three and four proteins.

### Example 2. Results

### Example 2.1. Individual Marker Results

Different metrics to evaluate the individual proteins were determined, also perming the following comparisons: CRC/AA vs CTL. Table 3 and Table 4 show these metrics for individual proteins, including p-value from Wilcoxon test (p.Wilc), area under the ROC curve (AUC), and Sensitivity (Sens.) and Specificity (Spec.) values, computed in the cut-off point of the ROC curve with the best Youden's index.

It can be seen that TFF3, CYFRA21-1, Flt3L and AREG are significantly (p<0.05) different between CRC and CTL groups and their AUC are significantly different from 0.5 (as their 95% confidence interval do not include 0.5). In the case of AA group, CASP4 and CYFRA21-1 also shows statistically differences compared to CTL group.

TABLE 3
CRC

| Marker | pWilcoxon | AUC | Sens | Spec |
|---|---|---|---|---|
| TFF3 | 0 | 0.751 (0.663-0.839) | 80 | 63.33 |
| | | | 78.33 | 65 |
| CYFRA21-1 | 0 | 0.746 (0.66-0.833) | 68.33 | 68.33 |
| | | | 56.67 | 80 |
| Flt3L | 0.001 | 0.682 (0.583-0.781) | 66.67 | 73.33 |
| AREG | 0.025 | 0.619 (0.518-0.719) | 81.67 | 43.33 |
| CASP4 | 0.071 | 0.596 (0.493-0.698) | 35 | 86.67 |
| IGFBP2 | 0.095 | 0.589 (0.487-0.691) | 98.33 | 16.67 |
| | | | 95 | 20 |
| | | | 20 | 95 |
| ADAMDEC1 | 0.122 | 0.582 (0.479-0.685) | 71.67 | 48.33 |
| DKK3 | 0.224 | 0.564 (0.461-0.668) | 93.33 | 25 |
| FASL | 0.378 | 0.547 (0.442-0.651) | 56.67 | 56.67 |
| | | | 55 | 58.33 |
| | | | 28.33 | 85 |
| DCSIGNR | 0.379 | 0.547 (0.442-0.651) | 41.67 | 75 |
| ErbB4 | 0.419 | 0.543 (0.439-0.647) | 58.33 | 55 |
| | | | 41.67 | 71.67 |
| | | | 40 | 73.33 |
| CD163 | 0.624 | 0.526 (0.422-0.63) | 43.33 | 66.67 |
| HGFR | 0.696 | 0.521 (0.416-0.626) | 61.67 | 53.33 |
| CEA | 0.767 | 0.516 (0.411-0.621) | 25 | 88.33 |
| CD147 | 0.869 | 0.509 (0.404-0.613) | 45 | 65 |
| PKM2 | 0.904 | 0.507 (0.402-0.611) | 46.67 | 63.33 |

### Individual Performance Metrics in CRC

TABLE 4
AA

| Marker | pWilcoxon | AUC | | Sens | Spec |
|---|---|---|---|---|---|
| CASP4 | 0.011 | 0.635 | (0.536-0.734) | 73.33 | 50 |
| CYFRA21-1 | 0.019 | 0.625 | (0.524-0.725) | 81.67 | 43.33 |
| | | | | 80 | 45 |
| TFF3 | 0.061 | 0.599 | (0.497-0.702) | 70 | 55 |
| | | | | 68.33 | 56.67 |
| | | | | 66.67 | 58.33 |
| Flt3L | 0.099 | 0.588 | (0.485-0.69) | 61.67 | 58.33 |
| IGFBP2 | 0.187 | 0.57 | (0.466-0.674) | 75 | 45 |
| | | | | 73.33 | 46.67 |
| | | | | 71.67 | 48.33 |
| DCSIGNR | 0.243 | 0.562 | (0.458-0.665) | 50 | 65 |
| ErbB4 | 0.265 | 0.559 | (0.455-0.663) | 65 | 55 |
| CD163 | 0.356 | 0.549 | (0.445-0.653) | 26.67 | 90 |
| HGFR | 0.372 | 0.547 | (0.443-0.652) | 65 | 51.67 |
| AREG | 0.613 | 0.527 | (0.422-0.632) | 90 | 23.33 |
| | | | | 88.33 | 25 |
| ADAMDEC1 | 0.654 | 0.524 | (0.419-0.628) | 38.33 | 73.33 |
| DKK3 | 0.688 | 0.521 | (0.417-0.626) | 50 | 60 |
| | | | | 46.67 | 63.33 |
| | | | | 43.33 | 66.67 |
| | | | | 38.33 | 71.67 |
| CD147 | 0.854 | 0.51 | (0.405-0.614) | 61.67 | 48.33 |
| | | | | 60 | 50 |
| FASL | 0.856 | 0.51 | (0.405-0.614) | 65 | 43.33 |
| CEA | 0.914 | 0.494 | (0.389-0.599) | 60 | 53.33 |
| | | | | 58.33 | 55 |
| PKM2 | 0.998 | 0.5 | (0.395-0.605) | 63.33 | 48.33 |
| | | | | 60 | 51.67 |

### Individual Performance Metrics in AA

Additionally, there are statistical significances between early stage vs control (p value=0.00098), and early stage+AA vs control (p value=0.0048) indicating that TFF3 is a good marker for screening purposes (i.e. early detection).

### Example 2.2. Best Combinations of Biomarkers

With the aim of improving individual diagnostic capability, combinations of proteins have been explored. All possible combinations of two, three and four proteins have been analyzed.

We have used the cohort of 180 subjects for developing models with all possible combinations and we have used the validation cohort (n=92) to obtain validated performance metrics (AUC values, Sens, Spec, PPV and NPV).

Table 5, Table 6 and Table 7 show the AUC achieved for the combinations of two, three and four biomarkers respectively, discriminating CRC from CTL.

TABLE 5

| Combinations of two biomarkers CRC vs CTL | AUC |
|---|---|
| TFF3/Flt3L | 0.8672 |
| Flt3L/CYFRA21-1 | 0.8135 |
| TFF3/CYFRA21-1 | 0.8047 |
| HGFR/CYFRA21-1 | 0.8037 |
| CYFRA21-1/CD147 | 0.8027 |
| CYFRA21-1/IGFBP2 | 0.7988 |
| CD163/CYFRA21-1 | 0.7979 |
| CYFRA21-1/CEA | 0.7949 |
| TFF3/CD147 | 0.7871 |
| Flt3L/CEA | 0.7871 |
| TFF3/HGFR | 0.7813 |

TABLE 6

| Combinations of three biomarkers CRC vs CTL | AUC |
|---|---|
| TFF3/Flt3L/HGFR | 0.8672 |
| TFF3/Flt3L/IGFBP2 | 0.8652 |
| TFF3/Flt3L/CD147 | 0.8574 |
| TFF3/Flt3L/CD163 | 0.8535 |
| Flt3L/CYFRA21-1/CD147 | 0.8135 |
| TFF3/Flt3L/CYFRA21-l | 0.8105 |
| Flt3L/HGFR/CYFRA21-1 | 0.8105 |
| Flt3L/CYFRA21-1/IGFBP2 | 0.8105 |
| Flt3L/CD163/CYFRA21-1 | 0.8096 |
| Flt3L/CYFRA21-1/CEA | 0.8096 |
| TFF3/CYFRA21-1/CD147 | 0.8076 |
| TFF3/HGFR/CYFRA21-1 | 0.8047 |
| TFF3/CYFRA21-1/IGFBP2 | 0.8047 |
| TFF3/CD163/CYFRA21-1 | 0.8027 |
| HGFR/CYFRA21-1/CD147 | 0.8027 |
| HGFR/CYFRA21-1/IGFBP2 | 0.8018 |
| CD163/CYFRA21-1/IGFBP2 | 0.8018 |
| CYFRA21-1/IGFBP2/CD147 | 0.7998 |
| CD163/HGFR/CYFRA21-1 | 0.7988 |
| TFF3/CYFRA21-1/CEA | 0.7979 |
| CD163/CYFRA21-1/CEA | 0.7979 |
| CD163/CYFRA21-1/CD147 | 0.7979 |
| HGFR/CYFRA21-1/CEA | 0.7959 |
| CYFRA21-1/CEA/CD147 | 0.7949 |
| CYFRA21-1/IGFBP2/CEA | 0.7939 |
| TFF3/HGFR/CD147 | 0.7891 |
| TFF3/IGFBP2/CD147 | 0.7881 |
| Flt3L/CEA/CD147 | 0.7871 |
| Flt3L/IGFBP2/CEA | 0.7822 |
| HGFR/Flt3L/CEA | 0.7793 |
| CD163/CYFRA21-1/FASL | 0.7722 |
| AREG/HGFR/CYFRA21-1 | 0.7705 |

TABLE 7

| Combinations of four biomarkers CRC vs CTL | AUC |
|---|---|
| TFF3/Flt3L/HGFR/IGFBP2 | 0.8652 |
| TFF3/Flt3L/HGFR/CD147 | 0.8584 |
| TFF3/Flt3L/IGFBP2/CD147 | 0.8574 |
| TFF3/Flt3L/CD163/IGFBP2 | 0.8418 |
| TFF3/Flt3L/CD163/HGFR | 0.8389 |
| TFF3/Flt3L/CD163/CD147 | 0.8193 |
| TFF3/Flt3L/CYFRA21-1/CD147 | 0.8135 |
| TFF3/Flt3L/CYFRA21-1/IGFBP2 | 0.8135 |
| TFF3/Flt3L/CD163/CYFRA21-l | 0.8125 |
| Flt3L/CYFRA21-1/IGFBP2/CD147 | 0.8125 |
| Flt3L/CD163/CYFRA21-1/IGFBP2 | 0.8115 |
| TFF3/Flt3L/HGFR/CYFRA21-1 | 0.8105 |
| Flt3L/HGFR/CYFRA21-1/CD147 | 0.8105 |
| Flt3L/HGFR/CYFRA21-1/IGFBP2 | 0.8105 |
| TFF3/CYFRA21-1/FASL/CD147 | 0.8105 |
| Flt3L/CYFRA21-1/CEA/CD147 | 0.8096 |
| DKK3/CD163/CYFRA21-1/IGFBP2 | 0.8096 |
| TFF3/CYFRA21-1/IGFBP2/CD147 | 0.8086 |
| Flt3L/HGFR/CYFRA21-1/CEA | 0.8086 |
| Flt3L/CD163/CYFRA21-1/CD147 | 0.8076 |
| Flt3L/CD163/HGFR/CYFRA21-1 | 0.8066 |
| Flt3L/CYFRA21-1/IGFBP2/CEA | 0.8066 |
| TFF3/HGFR/CYFRA21-1/CD147 | 0.8057 |
| Flt3L/CD163/CYFRA21-1/CEA | 0.8057 |
| TFF3/Flt3L/CYFRA21-1/CEA | 0.8047 |
| TFF3/HGFR/CYFRA21-1/IGFBP2 | 0.8047 |
| TFF3/CD163/CYFRA21-1/CD147 | 0.8037 |
| TFF3/CD163/HGFR/CYFRA21-1 | 0.8037 |
| TFF3/CD163/CYFRA21-1/IGFBP2 | 0.8037 |
| Flt3L/IGFBP2/CEA/CD147 | 0.8027 |
| HGFR/CYFRA21-1/IGFBP2/CD147 | 0.8018 |
| TFF3/CYFRA21-1/CEA/PKM2 | 0.8008 |
| CD163/HGFR/CYFRA21-1/IGFBP2 | 0.7988 |
| CD163/HGFR/CYFRA21-1/CD147 | 0.7988 |
| CYFRA21-1/IGFBP2/FASL/CD147 | 0.7984 |
| CD163/CYFRA21-1/IGFBP2/CD147 | 0.7979 |
| CD163/HGFR/CYFRA21-1/CEA | 0.7979 |
| CD163/CYFRA21-1/CEA/CD147 | 0.7979 |
| CD163/CYFRA21-1/IGFBP2/CEA | 0.7969 |
| HGFR/CYFRA21-1/CEA/CD147 | 0.7959 |
| TFF3/CYFRA21-1/CEA/CD147 | 0.7949 |
| TFF3/HGFR/CYFRA21-1/CEA | 0.7949 |
| TFF3/CYFRA21-1/IGFBP2/CEA | 0.7949 |
| TFF3/CD163/CYFRA21-1/CEA | 0.7939 |
| HGFR/CYFRA21-1/IGFBP2/CEA | 0.7939 |
| CYFRA21-1/IGFBP2/CEA/CD147 | 0.7939 |
| TFF3/HGFR/IGFBP2/CD147 | 0.7891 |
| Flt3L/HGFR/CEA/CD147 | 0.7832 |
| CD163/CYFRA21-1/FASL/CD147 | 0.7792 |
| Flt3L/HGFR/IGFBP2/CEA | 0.7754 |
| CD163/CYFRA21-1/IGFBP2/FASL | 0.7571 |
| Flt3L/HGFR/IGFBP2/CD147 | 0.7568 |
| CD163/CYFRA21-1/CEA/FASL | 0.7560 |

Since the TFF3/Flt3L pair appears as the best combination of two proteins and it is also present among the top combinations of three and four markers in the CRC. vs. CTL comparison, we have explored its performance in discriminating AA from CTL.

We obtained an AUC of 0.5361 when using the pair TFF3/Flt3L for discriminating AA from CTL. We combined this pair with the rest of proteins to obtain combinations of three and four markers. Table 8 and Table 9 shows the AUC achieved for these combinations of three and four biomarkers respectively, discriminating AA vs CTL.

TABLE 8

| Combinations of three biomarkers AA vs CNT | AUC |
|---|---|
| TFF3/Flt3L/ADAMDEC1 | 0.6430 |
| TFF3/Flt3L/CYFRA21-1 | 0.6358 |
| TFF3/Flt3L/FASL | 0.6310 |

TABLE 9

| Combinations of four biomarkers AA vs CTL | AUC |
|---|---|
| TFF3/Flt3L/CD163/ADAMDEC1 | 0.6815 |
| TFF3/Flt3L/FASL/ADAMDEC1 | 0.6623 |
| TFF3/Flt3L/IGFBP2/ADAMDECl | 0.6454 |
| TFF3/Flt3L/AREG/DKK3 | 0.6418 |
| TFF3/Flt3L/CYFRA21-1/CEA | 0.6418 |
| TFF3/Flt3L/HGFR/CYFRA21-1 | 0.6394 |
| TFF3/Flt3L/CYFRA21-1/CD147 | 0.6370 |
| TFF3/Flt3L/HGFR/ADAMDEC1 | 0.6370 |
| TFF3/Flt3L/CEA/ADAMDEC1 | 0.6358 |
| TFF3/Flt3L/ADAMDEC1/CD147 | 0.6346 |
| TFF3/Flt3L/CYFRA21-1/IGFBP2 | 0.6334 |
| TFF3/Flt3L/FASL/CD147 | 0.6334 |
| TFF3/Flt3L/CEA/FASL | 0.6322 |
| TFF3/Flt3L/HGFR/FASL | 0.6322 |
| TFF3/Flt3L/IGFBP2/FASL | 0.6298 |
| TFF3/Flt3L/CD163/FASL | 0.6178 |
| TFF3/Flt3L/AREG/DCSIGNR | 0.6130 |
| TFF3/Flt3L/CD163/IGFBP2 | 0.6010 |

Based on their respective AUCs, the best models have been selected. Table 10 shows the best results for CRC and Table 11 shows the best results for AA. The metrics for the best combinations of proteins are included, comprising area under the ROC curve (AUC), Sensitivity (Sens.), Specificity (Spec.), and positive (PPV) and negative (NPV) predictive values computed in the cut-off point of the ROC curve with the best Youden's index.

TABLE 10

| | | | | | |
|---|---|---|---|---|---|
| Best combinations for CRC | AUC | Sens | Spec | PPV | NPV |

Best combination of two biomarkers

| | | | | | |
|---|---|---|---|---|---|
| TFF3/Flt3L | 0.8672 | 0.8438 | 0.7813 | 0.7941 | 0.8333 |

Best combination of three biomarkers

| | | | | | |
|---|---|---|---|---|---|
| TFF3/Flt3L/HGFR | 0.8672 | 0.8438 | 0.7813 | 0.7941 | 0.8333 |
| TFF3/Flt3L/IGFBP2 | 0.8652 | 0.8750 | 0.7500 | 0.7778 | 0.8571 |
| TFF3/Flt3L/CD147 | 0.8574 | 0.9688 | 0.5625 | 0.6889 | 0.9474 |
| TFF3/Flt3L/CD163 | 0.8535 | 0.8125 | 0.7813 | 0.7879 | 0.8065 |
| TFF3/Flt3L/CYFRA21-1 | 0.8105 | 0.9063 | 0.6563 | 0.7250 | 0.8750 |

Best combination of four biomarkers

| | | | | | |
|---|---|---|---|---|---|
| TFF3/Flt3L/HGFR/IGFBP2 | 0.8652 | 0.8750 | 0.7500 | 0.7778 | 0.8571 |
| TFF3/Flt3L/HGFR/CD147 | 0.8584 | 0.9688 | 0.5625 | 0.6889 | 0.9474 |
| TFF3/Flt3L/IGFBP2/CD147 | 0.8574 | 0.9688 | 0.5625 | 0.6889 | 0.9474 |
| TFF3/Flt3L/CD163/IGFBP2 | 0.8418 | 0.7500 | 0.8125 | 0.8000 | 0.7647 |
| TFF3/Flt3L/CD163/HGFR | 0.8389 | 0.8125 | 0.7500 | 0.7647 | 0.8000 |
| TFF3/Flt3L/CD163/CD147 | 0.8193 | 0.7813 | 0.7500 | 0.7576 | 0.7742 |
| TFF3/Flt3L/CYFRA21-1/CD147 | 0.8135 | 0.9063 | 0.6563 | 0.7250 | 0.8750 |
| TFF3/Flt3L/CYFRA21-1/IGFBP2 | 0.8135 | 0.9063 | 0.6563 | 0.7250 | 0.8750 |
| TFF3/Flt3L/CD163/CYFRA21-1 | 0.8125 | 0.9063 | 0.6563 | 0.7250 | 0.8750 |
| TFF3/Flt3L/HGFR/CYFRA21-1 | 0.8105 | 0.8438 | 0.7500 | 0.7714 | 0.8276 |
| TFF3/Flt3L/CYFRA21-1/CEA | 0.8047 | 0.9063 | 0.6563 | 0.7250 | 0.8750 |

TABLE 11

| | | | | | |
|---|---|---|---|---|---|
| Best combinations for AA | AUC | Sens | Spec | PPV | NPV |

Best combinations of three biomarkers

| | | | | | |
|---|---|---|---|---|---|
| TFF3/Flt3L/ADAMDEC1 | 0.6430 | 0.6538 | 0.5938 | 0.5667 | 0.6786 |
| TFF3/Flt3L/CYFRA21-l | 0.6358 | 0.5385 | 0.7500 | 0.6364 | 0.6667 |
| TFF3/Flt3L/FASL | 0.6310 | 0.7692 | 0.5625 | 0.5882 | 0.7500 |

Best combinations of four biomarkers

| | | | | | |
|---|---|---|---|---|---|
| TFF3/Flt3L/CD163/ADAMDEC1 | 0.6815 | 0.6538 | 0.7188 | 0.6538 | 0.7188 |
| TFF3/Flt3L/FASL/ADAMDEC1 | 0.6623 | 0.7308 | 0.6875 | 0.6552 | 0.7586 |
| TFF3/Flt3L/IGFBP2/ADAMDEC1 | 0.6454 | 0.6538 | 0.5938 | 0.5667 | 0.6786 |
| TFF3/Flt3L/AREG/DKK3 | 0.6418 | 0.5000 | 0.8438 | 0.7222 | 0.6750 |
| TFF3/Flt3L/CYFRA21-1/CEA | 0.6418 | 0.5385 | 0.7500 | 0.6364 | 0.6667 |
| TFF3/Flt3L/HGFR/CYFRA21-1 | 0.6394 | 0.5385 | 0.7500 | 0.6364 | 0.6667 |
| TFF3/Flt3L/CYFRA21-1/CD147 | 0.6370 | 0.5385 | 0.7500 | 0.6364 | 0.6667 |
| TFF3/Flt3L/HGFR/ADAMDEC1 | 0.6370 | 0.6538 | 0.5938 | 0.5667 | 0.6786 |
| TFF3/Flt3L/CEA/ADAMDEC1 | 0.6358 | 0.6538 | 0.5938 | 0.5667 | 0.6786 |
| TFF3/Flt3L/ADAMDEC1/CD147 | 0.6346 | 0.6538 | 0.5938 | 0.5667 | 0.6786 |
| TFF3/Flt3L/CYFRA21-1/IGFBP2 | 0.6334 | 0.5385 | 0.7500 | 0.6364 | 0.6667 |
| TFF3/Flt3L/FASL/CD147 | 0.6334 | 0.7308 | 0.5938 | 0.5938 | 0.7308 |
| TFF3/Flt3L/CEA/FASL | 0.6322 | 0.7308 | 0.5938 | 0.5938 | 0.7308 |
| TFF3/Flt3L/HGFR/FASL | 0.6322 | 0.7692 | 0.5625 | 0.5882 | 0.7500 |
| TFF3/Flt3L/IGFBP2/FASL | 0.6298 | 0.7308 | 0.5938 | 0.5938 | 0.7308 |
| TFF3/Flt3L/CD163/FASL | 0.6178 | 0.7308 | 0.5625 | 0.5758 | 0.7200 |
| TFF3/Flt3L/AREG/DCSIGNR | 0.6130 | 0.8846 | 0.4063 | 0.5476 | 0.8125 |
| TFF3/Flt3L/CD163/IGFBP2 | 0.6010 | 0.3846 | 0.8125 | 0.6250 | 0.6190 |

Table 12 has been designed to show the overlapping of the most important signatures claimed in the present invention for CRC. It is clearly shown that most of the best signatures comprise [TFF3 and Flt3L]

TABLE 12

| 1 | 2 | 3 | 4 | 5 | 6 | 7 | 8 | 9 | 10 | 11 | 12 | 13 | 14 | 15 | 16 | CRC |
|---|---|---|---|---|---|---|---|---|---|---|---|---|---|---|---|---|
| X | | X | | | | | | | | | | | | | | 0.8672 |
| X | | X | | | | | | | | | | X | | | | 0.8672 |
| X | | X | | | X | | | | | | | | | | | 0.8652 |
| X | | X | | | X | | | | | | | X | | | | 0.8652 |

1) TFF3,
2) CYFRA21-1,
3) Flt3L,
4) AREG,
5) CASP4,
6) IGFBP2,
7) ADAMDEC1,
8) DKK3,
9) FASL,
10) DCSIGNR,
11) ErbB4,
12) CD163,
13) HGFR,
14) CEA,
15) CD147 and
16) PKM2

Table 13 has been designed to show the overlapping of the most important signatures claimed in the present invention for AA. It is clearly shown that [TFF3 and CYFRA21-1] is also included in combination of biomarkers showing a good performance for AA.

TABLE 13

| 1 | 2 | 3 | 4 | 5 | 6 | 7 | 8 | 9 | 10 | 11 | 12 | 13 | 14 | 15 | 16 | CRC |
|---|---|---|---|---|---|---|---|---|---|---|---|---|---|---|---|---|
| X | | X | | | | X | | | | | X | | | | | 0.6815 |
| X | | X | | | | X | | X | | | | | | | | 0.6623 |
| X | | X | | | X | X | | | | | | | | | | 0.6454 |
| X | | X | | | | X | | | | | | | | | | 0.6430 |

1) TFF3,
2) CYFRA21-1,
3) Flt3L,
4) AREG,
5) CASP4,
6) IGFBP2,
7) ADAMDEC1,
8) DKK3,
9) FASL,
10) DCSIGNR,
11) ErbB4,
12) CD163,
13) HGFR,
14) CEA,
15) CD147 and
16) PKM2

1. An in vitro method for screening for colorectal cancer or a precancerous stage thereof comprising:
a) measuring the concentration level of at least protein TFF3 (Trefoil factor 3), in a plasma sample obtained from a subject,
b) determining if the concentration level of TFF3 is statistically higher than a reference concentration level of TFF3 measured in healthy control subjects, and
c) identifying said subject as one having colorectal cancer or a precancerous stage thereof when the measured concentration level of TFF3 is statistically higher than said measured reference concentration level.

2-13. (canceled)

14. The in vitro method according to claim 1, comprising:
a) measuring the concentration levels of at least proteins TFF3 and Flt3L (Fms-related tyrosine kinase 3 ligand), in plasma sample obtained from the subject,
b) determining if the concentration levels of TFF3 and Flt3L are statistically higher than the concentration levels of TFF3 and Flt3L measured in healthy control subjects, and
c) identifying said subject as one having colorectal cancer or a precancerous stage thereof when the measured concentration levels of TFF3 and Flt3L are statistically higher than said measured reference concentration levels.

15. The in vitro method, according to claim 1, comprising:
a) measuring the concentration level of the proteins from at least one of the following groups of proteins: TFF3, Flt3L and HGFR; TFF3, Flt3L and IGFBP2; TFF3, Flt3L and CD147; TFF3, Flt3L and CD163; TFF3, Flt3L and CYFRA21-1; TFF3, Flt3L and ADAMDEC1; TFF3, Flt3L and FASL; TFF3, Flt3L, HGFR and IGFBP2; TFF3, Flt3L, HGFR and CD147; TFF3, Flt3L, IGFBP2 and CD147; TFF3, Flt3L, CD163 and IGFBP2; TFF3, Flt3L, CD163 and HGFR; TFF3, Flt3L, CD163 and CD147; TFF3, Flt3L, CYFRA21-1 and CD147; TFF3, Flt3L, CYFRA21-1 and IGFBP2; TFF3, Flt3L, CD163, and CYFRA21-1; TFF3, Flt3L, HGFR, and CYFRA21-1; or TFF3, Flt3L, CYFRA21-1, and CEA in a plasma sample obtained from the subject;
b) determining if the concentration levels of the proteins in any one or more of the groups of proteins in a) is statistically higher than the concentration levels of the same groups of proteins measured in healthy control subjects; and
c) identifying said subject as one having colorectal cancer or a precancerous stage thereof when the concentration levels of the proteins measured in any one or more of the groups of proteins in a) are statistically higher than said measured reference concentration levels.

16. The in vitro method according to claim 3, further comprising processing the measured concentration values of the proteins to obtain a risk score and identifying said subject as one having colorectal cancer or a precancerous stage thereof when the concentration level of the proteins measured are statistically higher than said measured reference concentration levels.

17. The in vitro method according to claim 1, wherein the pre-cancerous stage of colorectal cancer is advanced colorectal adenoma.

18. The in vitro method according to claim 1, further comprising confirming the identification of said subject as one having colorectal cancer or a precancerous stage thereof by an imaging technique.

19. A kit for screening for colorectal cancer or a pre-cancerous stage thereof comprising:
a) reagents or tools suitable for obtaining a plasma sample from a subject, and
b) reagents or tools suitable for determining the concentration level of TFF3 (Trefoil factor 3) in said plasma sample.

20. The kit according to claim 19, further comprising reagents or tools suitable for determining the concentration level of Flt3L (Fms-related tyrosine kinase 3 ligand) in said plasma sample.

21. The kit according to claim 19, comprising reagents or tools for determining the concentration levels of the proteins from at least one of the following groups of proteins: TFF3, Flt3L and HGFR; TFF3, Flt3L and IGFBP2; TFF3, Flt3L and CD147; TFF3, Flt3L and CD163; TFF3, Flt3L and CYFRA21-1; TFF3, Flt3L and ADAMDEC1; TFF3, Flt3L and FASL; TFF3, Flt3L, HGFR and IGFBP2; TFF3, Flt3L, HGFR and CD147; TFF3, Flt3L, IGFBP2 and CD147; TFF3, Flt3L, CD163 and IGFBP2; TFF3, Flt3L, CD163 and HGFR; TFF3, Flt3L, CD163 and CD147; TFF3, Flt3L, CYFRA21-1 and CD147; TFF3, Flt3L, CYFRA21-1 and IGFBP2; TFF3, Flt3L, CD163, and CYFRA21-1; TFF3, Flt3L, HGFR, and CYFRA21-1; or TFF3, Flt3L, CYFRA21-1, and CEA in a plasma sample obtained from the subject.
